(19) United States
(12) Patent Application Publication
(10) Pub. No.: US 2024/0325197 A1
(43) Pub. Date: Oct. 3, 2024

(54) PHACOEMULSIFICATION NEEDLE

(71) Applicant: RAICO International, LLC, Willowbrook, IL (US)

(72) Inventors: Ravi Nallakrishnan, Willowbrook, IL (US); Takayuki Akahoshi, Tokyo (JP); Arulmozhi Varman, Chennai (IN)

(21) Appl. No.: 18/293,154

(22) PCT Filed: Aug. 11, 2022
(86) PCT No.: PCT/US2022/040023

§ 371 Date: Jan. 29, 2024

Related U.S. Application Data
(60) Provisional application No. 63/232,447, filed on Aug. 12, 2021.

(51) Int. Cl. A61F 9/007; A61B 17/00; A61B 17/32; A61B 17/34

(52) U.S. Cl. CPC A61F 9/00754; A61F 9/00745; A61B 2017/00526; A61B 2017/320064; A61B 2017/3454

(57) ABSTRACT

A surgical instrument assembly includes a phacoemulsification needle (10) for use with a phacoemulsification handpiece. The needle (10) includes a needle shaft portion (14) defining a longitudinal axis (12) and having an internal aspiration passage (16). The needle (10) further includes an emulsification tip (20) joined to a distal end of the needle shaft portion (14). In one form, the tip (20) has a five side walls (22, 24, 26, 28, 30) defining a perimeter of an open mouth (34) communicating with said aspiration passage (16), three of said side walls (22, 24, 26) defining a first thickness (T1), the remaining two of said side walls (28, 30) converging at a location in a vertical plane (P1) that extends through said longitudinal shaft axis (12) and having a second thickness (T2) that is greater than said first thickness (T1).

### PRIORITY

This application claims priority of U.S. Provisional Patent Application Ser. No. 63/232,447, filed Aug. 12, 2021, the entire contents of which is incorporated herein by reference.

### FIELD OF THE INVENTION

The present invention relates generally to surgical instruments used in ophthalmological surgery and, more particularly, to an improved phacoemulsification needle which is particularly suited for use with an associated ultrasonic vibratory surgical handpiece to facilitate efficient operation and use thereof.

### BACKGROUND OF THE INVENTION

Phacoemulsification has come to be a technique of choice for the removal of damaged or diseased lenses from the eye. Commonly, such surgery is called for when a patient develops cataracts, a condition in which a portion of the eye lens becomes hard and opaque. Unless the damaged lens is removed and replaced with a properly selected artificial lens, blindness or severely impaired vision will result.

Phacoemulsification is the use of ultrasonic energy to emulsify the damaged lens and aspirate the resulting lens particles from the eye. One of the most significant advantages of the use of phacoemulsification is that the apparatus itself is small and can fit through a relatively small incision, resulting in less fluid leakage from the eye capsule and shorter patient recovery times. It is desirable to limit the amount of ultrasonic energy used as much as possible in order to minimize the risk of damage to eye tissue. Often, the lens nucleus (the hardest portion of the lens) is chopped or split into smaller pieces prior to or during phacoemulsification. Smaller pieces require less energy to emulsify, and this shortens the time during which ultrasonic energy is actually being created by the phacoemulsification apparatus.

Commonly, an infusion sleeve is mounted around the needle to supply irrigating liquids to the eye in order to maintain positive pressure in the eye as the emulsified lens nucleus and fluids are aspirated through the hollow lens.

It is extremely important to properly infuse liquid during such surgery. Maintaining a sufficient amount of liquid prevents collapse of certain tissues within the eye and attendant injury or damage to delicate eye structures. As an example, endothelial cells can easily be damaged during such collapse and this damage is permanent because these cells do not regenerate. One of the benefits of using as small in incision as possible during such surgery is the minimization of leakage of liquid during and after surgery to help prevent tissue collapse, faster healing time, and decreased post-operative astigmatism.

U.S. Pat. No. 7,601,136, hereby incorporated by reference, discloses a phacoemulsification needle and sleeve assembly.

Many phacoemulsification needles and tips are designed for use with handpieces that vibrate the needle longitudinally at relatively low frequencies. In addition to longitudinal vibration, certain handpieces impart a torsional motion to the needle at an oscillation frequency of about 100 cycles per second. There are also handpieces that provide torsional oscillation of the phacoemulsification tip at frequencies of about 32,000 cycles per second.

Use of the torsional-type handpiece has called for phacoemulsification needle tip designs differing from those used with the longitudinal-type handpiece. For example, needles have been designed with tips that are shaped, swaged and angled to take advantage of the needle motion created by the handpiece.

Many surgeons favor phacoemulsification needles having the straight tip design commonly used with longitudinal handpieces. The great majority of surgeons use longitudinal handpieces rather than the torsional handpieces, often because torsional phacoemulsification equipment is more expensive than longitudinal equipment, and thus these surgeons find themselves unable to take advantage of the enhanced phacoemulsification results claimed by the torsional phacoemulsification systems.

U.S. Pat. No. 10,952,895, hereby incorporated by reference in its entirety, discloses a needle tip in an off-axis position relative to the axis of the aspiration passage extending through the needle body causes eccentric motion or “wobble” during torsional phacoemulsification and improves the efficiency of phacoemulsification while retaining the straight-tip configuration. It has also been found that forming the tip in such an off-axis position also increases the efficiency of phacoemulsification when using a longitudinal handpiece.

Use of an off-axis tip with a longitudinal hand piece appears to desirably create a hybrid type of phacoemulsification motion without using the more complex and expensive torsional phacoemulsification apparatus. The eccentric or wobble type of motion can be imparted to a phacoemulsification needle with no flare at the tip by forming the central aspiration passage within the needle body in an off-axis position. Similar results can be obtained using a straight phacoemulsification needle having an aspiration passage that is formed with a cross-sectional configuration different than the cross-sectional configuration of the needle body itself. These results will be further amplified if the passage is also placed off-axis.

The present invention is directed to an improved phacoemulsification surgical instrument assembly, wherein an improved phacoemulsification needle having an improved tip configuration to promote improved emulsification efficiency, improved aspiration, and/or the minimization of the transmission of thermal energy to the site during a procedure.

### SUMMARY OF THE INVENTION

The present invention is directed to an improved phacoemulsification needle which is particularly suited for use with an associated vibratory surgical handpiece, wherein the handpiece may be configured for torsional (i.e., rotational) ultrasonic movement, as well as linear or longitudinal movement, elliptical, or blended movement, etc.

In accordance with one broad form of the present invention, the phacoemulsification needle includes a needle shaft portion defining a longitudinal axis and having an internal aspiration passage. The needle includes an emulsification tip joined to a distal end of the needle shaft portion. The emulsification tip has five side walls defining a perimeter of an open mouth communicating with the aspiration passage. Three of the side walls define a first thickness, while the remaining two of the side walls converge at a location in a vertical plane that extends through the longitudinal shaft axis and have a second thickness that is greater than the first thickness. Preferably, the first thickness is about 100 microns. Further, the second thickness is preferably between about 150 microns and about 170 microns.

In one preferred form of the present invention, the five side define a substantially pentagonal perimeter having symmetry about the aforementioned plane.

In another preferred form of the present invention, the emulsification tip has a distal edge disposed at an acute angle to a plane extending normally through the longitudinal axis of the needle shaft portion. Preferably, the acute angle is between about 20 and 40 degrees, and more preferably about 30 degrees.

In yet another preferred form of the present invention, adjacent ones of the five side walls converge at convex corners for enhanced safety during operation of the needle.

In yet another preferred form of the present invention, the open mouth defines a longitudinal mouth axis that is parallel to, and offset from, the longitudinal shaft axis.

According to one preferred form of the present invention, the emulsification tip extends a length along the longitudinal shaft axis between about 1.95 mm and about 2.05 mm.

In accordance with one broad form of the present invention, the phacoemulsification needle includes a needle shaft portion defining a longitudinal axis and having an internal aspiration passage. The needle includes an emulsification tip joined to a distal end of the needle shaft portion. The emulsification tip has five side walls defining a perimeter of an open mouth communicating with the aspiration passage. Two of the side walls converge in a rounded corner at a location in a vertical plane that extends through the longitudinal shaft axis. Preferably, the side walls all have the same nominal thickness, which is preferably about 100 microns. Further, the second thickness is preferably between about 150 microns and about 170 microns.

In accordance with another broad form of the present invention, the phacoemulsification needle includes a needle shaft portion defining a longitudinal axis and having an internal aspiration passage. The needle includes an emulsification tip joined to a distal end of the needle shaft portion. The emulsification tip has six side walls defining a perimeter of an open mouth communicating with the aspiration passage. A first pair of the side walls is substantially parallel to one another. A second pair of the side walls is substantially parallel to one another. A third pair of the side walls extend from one of the first pair of the side walls to one of the second pair of the side walls. Preferably, the side walls all have the same nominal thickness, which is preferably about 100 microns. Further, the second thickness is preferably between about 150 microns and about 170 microns.

According to one preferred form of the present invention, the open mouth is substantially square.

In another preferred form of the present invention, the first pair of side walls defines a first thickness and one of the second pair of the side walls defines a second thickness that is substantially greater than the first thickness.

In accordance with one broad form of the present invention, the phacoemulsification needle includes a needle shaft portion defining a longitudinal axis and having an internal aspiration passage. The needle includes an emulsification tip joined to a distal end of the needle shaft portion. The emulsification tip has four side walls defining a perimeter of an open mouth communicating with the aspiration passage. The open mouth has a pentagonal shape.

In one preferred form of the present invention, the open mouth defines a longitudinal mouth axis that is parallel to, and offset from, the longitudinal shaft axis. Furthermore, the open mouth is symmetric about a plane extending through both of the longitudinal mouth axis and the longitudinal shaft axis.

In another preferred form of the present invention, the open mouth defines a longitudinal mouth axis that is parallel to, and offset from, the longitudinal shaft axis. Furthermore, the open mouth is asymmetric about a plane extending through both of the longitudinal mouth axis and the longitudinal shaft axis.

According to one preferred form of the present invention, a proximal end of the needle shaft portion is provided with means for being removably attached to a vibratory handpiece. Preferably, the needle is in combination with a vibratory handpiece.

In accordance with another broad form of the present invention, the phacoemulsification needle includes a needle shaft portion defining a longitudinal axis and having an internal aspiration passage. The needle includes an emulsification tip joined to a distal end of the needle shaft portion having five side walls defining a perimeter of an open mouth communicating with the aspiration passage. The aspiration passage has a pentagonal cross-sectional shape in a plane normal to the longitudinal shaft axis.

In yet another broad form of the present invention, the phacoemulsification needle includes a needle shaft portion defining a longitudinal axis and having an internal aspiration passage. The needle includes an emulsification tip joined to a distal end of the needle shaft portion having five side walls defining a perimeter of an open mouth communicating with the aspiration passage. The emulsification tip has an intermediate passage formed therein that is offset from the longitudinal shaft axis. Preferably, the intermediate passage is semi-circular and is centered along a vertical plane extending through the longitudinal shaft axis.

In still another broad form of the present invention, the phacoemulsification needle includes a needle shaft portion defining a longitudinal axis and having an internal aspiration passage. The needle includes an emulsification tip joined to a distal end of the needle shaft portion having five side walls defining a perimeter of an open mouth communicating with the aspiration passage. The lower or bottom walls of the tip extend outwardly at a first acute angle α1 relative to a vertical plane extending normal to the central axis central axis and the upper walls 22I and 26I extend at a lesser acute angle compared to the angle α2 relative to the plane P2.

### BRIEF DESCRIPTION OF THE DRAWINGS

In the accompanying drawings forming part of the specification, in which like numerals are employed to designate like parts throughout the same,

FIG. 1 is an isometric view, from the front and below, of a first illustrated embodiment of a phacoemulsification surgical needle of the present invention;

FIG. 2 is a right-side elevational view of the instrument shown in FIG. 1;

FIG. 3 is a front elevational view of the instrument shown in FIG. 1;

FIG. 4 is a cross-sectional view of the instrument shown in FIG. 1, taken along a vertical plane 4-4 in FIG. 3 extending through a central longitudinal axis of the instrument;

FIG. 5 is a greatly enlarged, fragmentary, right side elevational view of only the tip portion of the instrument shown in FIG. 1;

FIG. 6 is a greatly enlarged, fragmentary, cross-sectional view of only the tip portion of the instrument shown in FIG. 4;

FIG. 7 is another front elevational view of the instrument shown in FIG. 1;

FIG. 8 is a greatly enlarged, fragmentary, isometric view, from the front and right-side, of only the distal portion of the instrument shown in FIG. 1;

FIG. 9 is an isometric view, from the front and above, of the instrument shown in FIG. 1;

FIG. 10 is a greatly enlarged, fragmentary, cross-sectional view, of only the distal portions of the instrument shown in FIG. 1;

FIG. 11 is a greatly enlarged, isometric wireframe view, taken from the front and above, of only the distal portions of the instrument shown in FIG. 1;

FIG. 12 is an isometric wireframe view, taken from the right side and above, of the instrument shown in FIG. 1;

FIG. 13 is a cross-sectional view of the instrument shown in FIG. 1;

FIG. 14 is another cross-sectional view of the instrument shown in FIG. 1;

FIG. 15 is a greatly-enlarged, fragmentary, cross-sectional view of the tip portion of the instrument shown in FIG. 1;

FIG. 16 is a front elevational view of a second illustrated embodiment of a phacoemulsification surgical needle of the present invention;

FIG. 17 is a right-side elevational view of the instrument shown in FIG. 16;

FIG. 18 is an isometric view, taken from below and behind, of the instrument shown in FIG. 16;

FIG. 19 is a greatly-enlarged, fragmentary, right side elevational view of the tip portion of the instrument shown in FIG. 17;

FIG. 20 is a cross-sectional view of the instrument shown in FIG. 16, taken along a vertical plane 20-20 in FIG. 16 extending through a central longitudinal axis of the instrument;

FIG. 21 is a greatly enlarged, fragmentary, cross-sectional view of only the tip portion of the instrument shown in FIG. 20;

FIG. 22 is another front elevational view of the instrument shown in FIG. 16;

FIG. 23 is a fragmentary, greatly enlarged, isometric view, taken from above and the right side, of the instrument shown in FIG. 16;

FIG. 24 is an isometric view, taken from above and the front side, of the instrument shown in FIG. 16;

FIG. 25 is a greatly enlarged, fragmentary, cross-sectional view, of the instrument shown in FIG. 16;

FIG. 26 is a greatly enlarged, fragmentary, wireframe isometric view, of the instrument shown in FIG. 16;

FIG. 27 is a wireframe isometric view, taken from above and the right side, of the instrument shown in FIG. 16;

FIG. 28 is another isometric view, taken from the front and right-side, of the instrument shown in FIG. 16;

FIG. 29 is a greatly enlarged, fragmentary, isometric view, taken from the front and right-side, of the instrument shown in FIG. 16;

FIG. 30 is a fragmentary, greatly enlarged, cross-sectional view of the instrument shown in FIG. 16;

FIG. 31 is an isometric view, taken from the front and right-side, of a third illustrated embodiment of a phacoemulsification surgical needle of the present invention;

FIG. 32 is a fragmentary, greatly enlarged, isometric view, taken from the front and left-side, of the instrument shown in FIG. 31;

FIG. 33 is a front elevational view of the instrument shown in FIG. 31;

FIG. 34 is a fragmentary, isometric view, taken from the front and left-side, of the instrument shown in FIG. 31;

FIG. 35 is another fragmentary, isometric view, taken from the front and left-side, of the instrument shown in FIG. 31;

FIG. 36 is a fragmentary, isometric view, taken from the front and left-side, of another embodiment of the instrument shown in FIG. 31;

FIG. 37 is another fragmentary, isometric view, taken from the front and left-side, of the instrument shown in FIG. 36;

FIG. 38 is a greatly enlarged, fragmentary, front elevational view of the instrument shown in FIG. 31;

FIG. 39 is a greatly enlarged, fragmentary, isometric view, taken from the front and left-side, of the instrument shown in FIG. 31;

FIG. 40 is a greatly enlarged, fragmentary, front elevational view, of a fourth illustrated embodiment of a phacoemulsification surgical needle of the present invention;

FIG. 41 is a greatly enlarged, fragmentary, isometric view, taken from the front and left-side, of the instrument shown in FIG. 40;

FIG. 42 is a greatly enlarged, fragmentary, front elevational view, of a fifth illustrated embodiment of a phacoemulsification surgical needle of the present invention;

FIG. 43 is a greatly enlarged, fragmentary, front elevational view, of a sixth illustrated embodiment of a phacoemulsification surgical needle of the present invention;

FIG. 44 is a greatly enlarged, fragmentary, isometric view, taken from the front and left-side, of the instrument shown in FIG. 43;

FIG. 45 is a greatly enlarged, fragmentary, front elevational view, of a seventh illustrated embodiment of a phacoemulsification surgical needle of the present invention;

FIG. 46 is a greatly enlarged, fragmentary, isometric view, taken from the front and right-side, of the instrument shown in FIG. 45;

FIG. 47 is a greatly enlarged, fragmentary, front elevational view of an eighth illustrated embodiment of a phacoemulsification surgical needle of the present invention;

FIG. 47A is a greatly enlarged, fragmentary, front elevational view of a variation of the eighth illustrated embodiment of FIG. 47;

FIG. 48 is a cross-sectional view of the instrument shown in FIG. 47, taken along a vertical plane P1 in FIG. 47 extending through a central longitudinal axis of the instrument;

FIG. 49 is a greatly enlarged, front elevational view of a ninth illustrated embodiment of a phacoemulsification surgical needle of the present invention;

FIG. 49A is a greatly enlarged, front elevational view of a variation of the ninth illustrated embodiment of FIG. 49;

FIG. 50 is a greatly enlarged, fragmentary, right side elevational view of a tenth illustrated embodiment of a phacoemulsification surgical needle of the present invention;

FIG. 51 is a greatly enlarged, fragmentary, detailed view of the distal portion of the needle tip that is circled in FIG. 50;

FIG. 52 is a greatly enlarged, fragmentary, detailed view of the circled distal portion of the needle tip in FIG. 50, however, FIG. 52 illustrates a variation of the embodiment having a rounded distalmost end of the tip portion;

FIG. 53 is a greatly enlarged, front elevational view of the surgical needle of FIG. 52;

FIG. 54 is a greatly enlarged, fragmentary, top plan view of the surgical needle of FIG. 52;

FIG. 55 is a greatly enlarged, fragmentary, cross-sectional view of a variation of the instrument shown in FIG. 50, taken along a central vertical plane extending through a central longitudinal axis of the instrument, and FIG. 55 shows the instrument with a downward bend located proximate to the tip portion; and

FIG. 56 is a greatly enlarged, fragmentary, side elevation view of another variation of the instrument shown in FIG. 50, and FIG. 56 illustrates some internal features of the instrument in hidden line.

### DETAILED DESCRIPTION OF THE PREFERRED EMBODIMENTS

While the present invention is susceptible of embodiment in various forms, there are shown in the drawings and will hereinafter be described the presently preferred embodiments, with the understanding that the present disclosure should be considered as an exemplification of the invention, and is not intended to limit the broadest forms of the invention to only the specific embodiments illustrated.

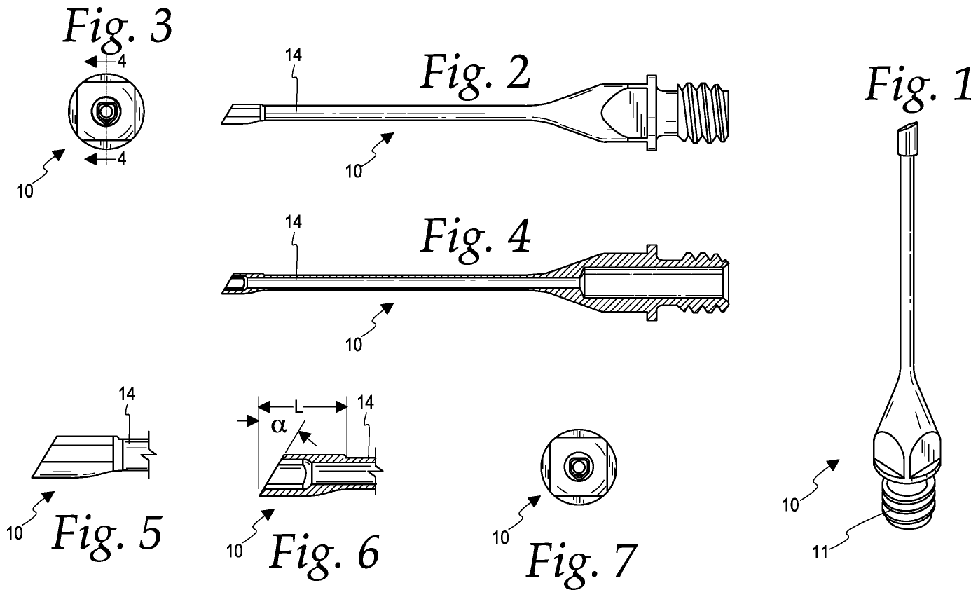

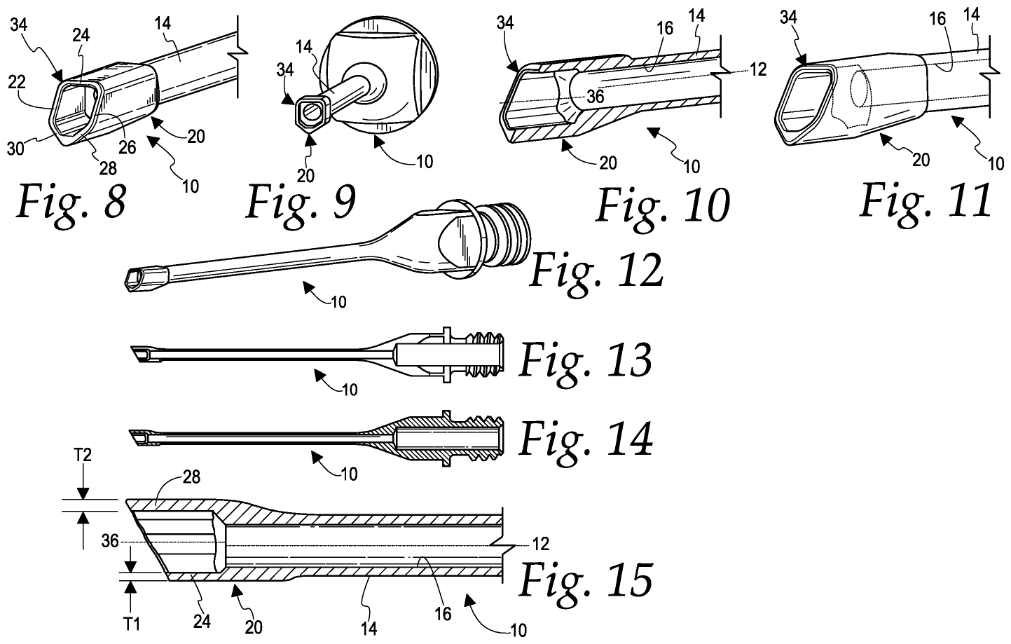

A first illustrated embodiment of a surgical instrument or phacoemulsification needle 10 according to the present invention is shown in FIGS. 1-15, wherein the needle 10 includes an elongate needle shaft portion 14 having a threaded proximal end 11 (FIG. 1) for being connected to mating threads in a vibratory handpiece and defining a longitudinal axis 12 (FIG. 15). The shaft portion 14 defines an internal aspiration passage 16 (FIG. 15), through which aspiration is effected during phacoemulsification. Only a distal portion of the needle 10 is illustrated in many of the accompanying figures, and it will be understood that the proximal end 11 of the needles disclosed herein may have a variety of means or structures suited for being attached to a vibratory hand piece, such as by mating screw threads, clamps, locks, friction fitting, etc. Suitable proximal end structures of needles are described in U.S. Pat. No. 8,764,782, the entirety of which is incorporated herein by reference.

With reference to FIG. 8, the present phacoemulsification needle 10 further comprises an emulsification tip 20 joined to a distal end of the needle shaft portion 14, opposite of the proximal end 11. Notably, the emulsification tip 20 has a generally, pentagonal cross-sectional configuration, and includes five side walls, or simply referred to hereinafter as “walls”, 22, 24, 26, 28, 30 defining a perimeter of an open mouth 34. The open mouth 34 of the tip 20 connects to the aspiration passage 16 of the needle shaft portion 14 to facilitate aspiration of emulsified tissues through the needle 10 when subjected to a vacuum.

As can be seen in FIG. 15, the present emulsification needle 10 desirably includes a first thickness T1 of at least one of the walls 22, 24, 26, and a substantially greater second thickness T2 of at least one of the other walls 28, 30, which effect phacoemulsification attendant to torsional, longitudinal, elliptical, and/or blended ultrasonic movement of the needle 10. Preferably, each of the walls 28, 30 converge at a location in a vertical plane “P1” that extends through the longitudinal shaft axis 12 and have a symmetry about this vertical plane “P1” (as can be seen in FIG. 3). More preferably, this plane “P1” extends through the bottom of the needle tip portion 20. Preferably, each of the walls 22, 24, 26 have the same nominal thickness T1 of about 100 microns, and each of the converging lower or bottom walls 28, 30 have the same nominal thickness T2 of about 150-170 microns. In the illustrated preferred embodiment of the instrument 10, adjacent ones of the walls 22, 24, 26, 28, 30 of the tip portion 20, defining the mouth 34, advantageously connect in rounded, convexly-curved corners for enhanced safety in the eye. With reference to FIG. 6, the emulsification tip 20 preferably extends a length “L” along the longitudinal shaft axis 12 between about 1.95 mm and about 2.05 mm.

With reference to FIG. 6, the emulsification tip 20 has a distal edge disposed at an acute angle α of between about 20 and 40 degrees, more preferably about 30 degrees, relative to the plane “P2” extending normal to the longitudinal axis 12 of the needle shaft portion 14. This arrangement facilitates efficient use, and is particularly suitable for use with an ultrasonic instrument configured to move ultrasonically in linear and/or torsional modes.

With reference to FIG. 15, the mouth 34 defines a longitudinal mouth axis 36 that is substantially parallel to, and offset from, the longitudinal axis 12 of the needle shaft portion 14, to enhance the emulsifying or chopping action of the thickened converging, bottom walls 28, 30 during vibratory operation of the needle 10. In some embodiments, not preferred or illustrated, the mouth axis 36 may be coaxial with the axis 12 of the needle body 14.

A desirable feature of the present phacoemulsification needle is the absence of sharp edges on the exterior of the needle 10. The forward, distal edges of the emulsification tip 20 are preferably rounded and smooth, without sharp edges. There are preferably no sharp edges on the outer periphery of the tip 20.

The specific configuration of the present needle 10 can be varied depending upon intended use. The needle shaft portion 14 can be straight as in the illustrated embodiments, or the needle shaft portion 14 may be bent to a Kelman-style configuration for effecting chopping during a surgical procedure. Alternatively, the needle shaft portion 14 can be bent to an Akahoshi-style (Reverse Kelman Bend) for pre-chopping during a procedure. Other bent configurations are contemplated.

With reference to FIGS. 5 and 8, the outer surface of the tip 20 may have a sandblasted finish to eliminate or at least reduce the potential for sharp edges for improved safety of the instrument 10 in the eye. Such sandblasting or other conventional or non-conventional finishing methods may be applied to the needle embodiments discussed below.

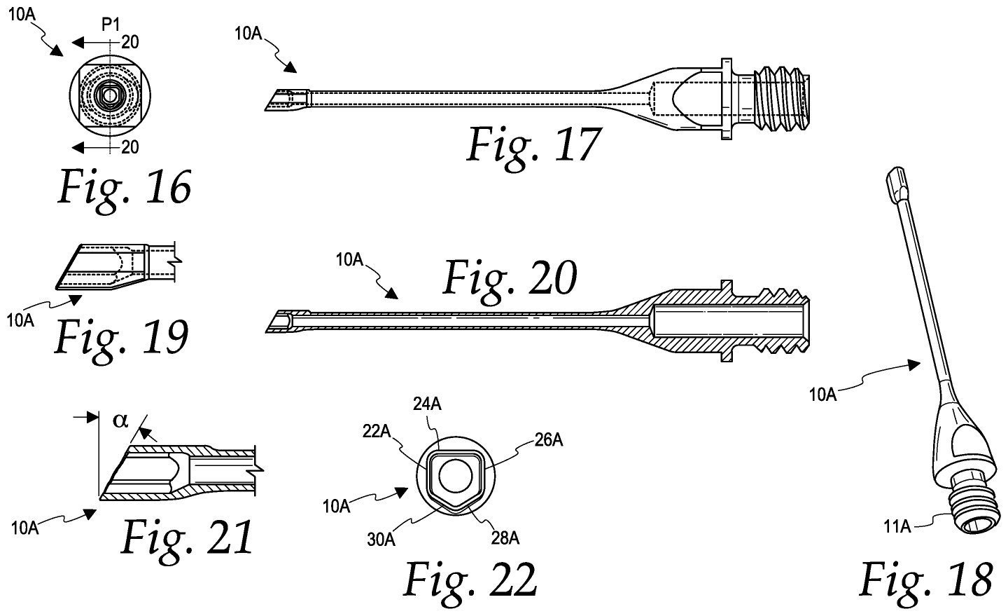

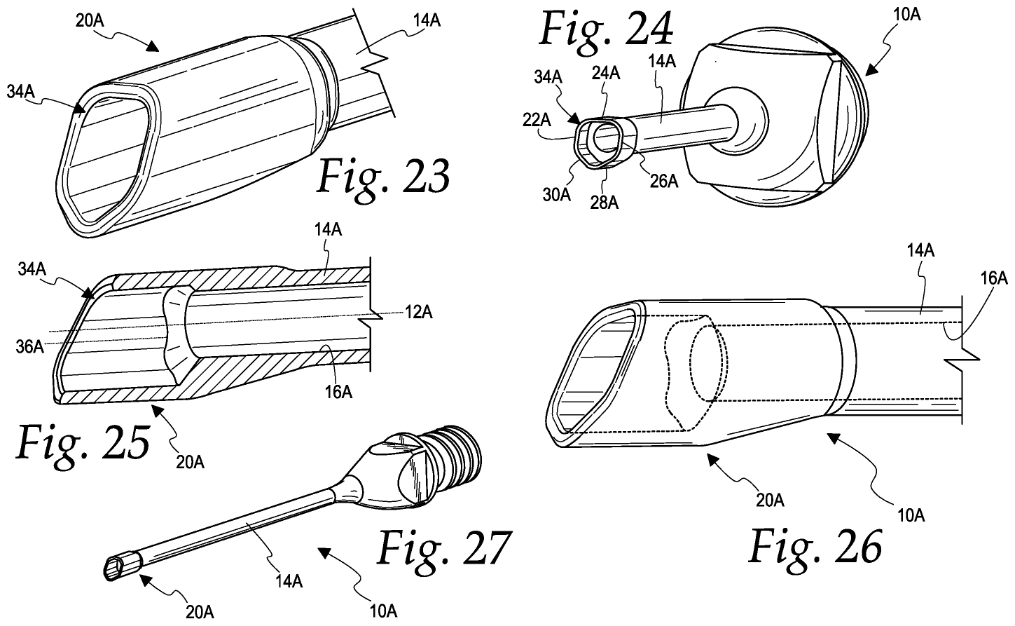

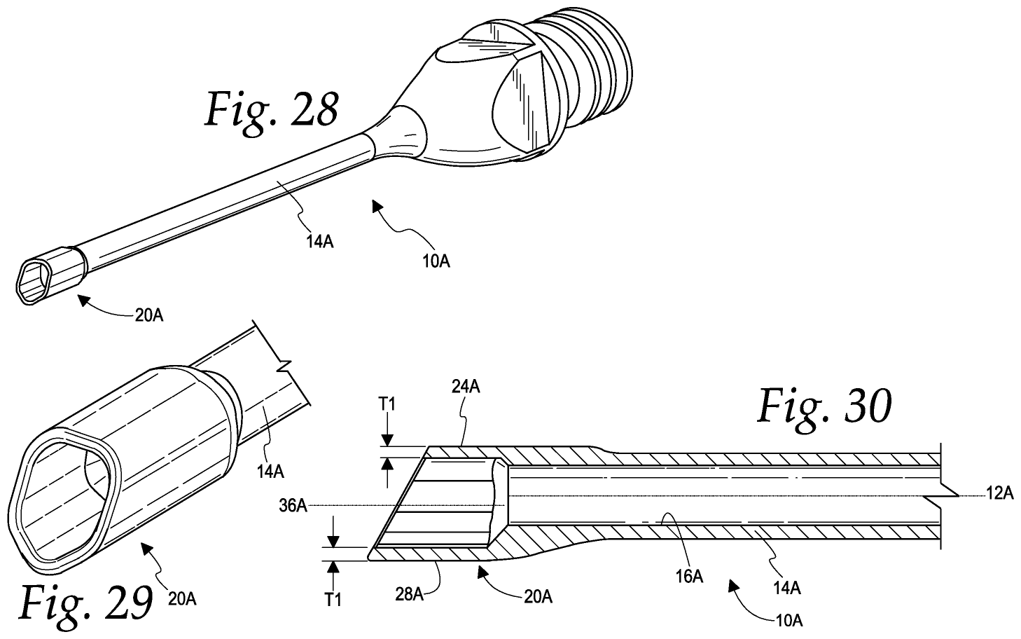

A second embodiment of a surgical needle according to the present invention is shown in FIGS. 16-30, designated by the numeral 10A, and functions similarly to the first illustrated embodiment of the needle 10 as previously described in FIGS. 1-15. The numbered features of the second embodiment of the needle 10A illustrated in FIGS. 16-30 are analogous to features of the first embodiment of the needle 10 that share the same number, but without the suffix “A”. The second embodiment of the surgical needle 10A differs from the aforementioned first illustrated embodiment in that the second embodiment includes an emulsification tip 20A with a generally, pentagonal cross-sectional configuration, and includes a five equal thickness side walls, or simply “walls”, 22A, 24A, 26A, 28A, 30A defining a perimeter of an open mouth 34A.

As can be seen in FIGS. 22 and 30, the walls 22A, 24A, 26A, 28A, 30A, share a nominal thickness T1 of about 100 microns. Preferably, each of the walls 28A, 30A converge at a location in a vertical plane “P1” that extends through the longitudinal shaft axis 12A and have a symmetry about this vertical plane “P1” (as can be seen in FIG. 16). More preferably, this plane “P1” extends through the bottom of the needle tip portion 20A. In the illustrated preferred embodiment of the instrument 10A, adjacent ones of the walls 22A, 24A, 26A, 28A, 30A of the tip portion 20A, defining the mouth 34A, advantageously connect in rounded, convexly-curved corners for enhanced safety in the eye. Preferably, the corners are rounded between R0.2-R0.50. In one non-preferred embodiment, the one or more of the corners are 90-degree bends.

The second illustrated embodiment of the needle 10A may provide a more efficient phacoemulsification as compared to the first illustrated embodiment of the needle when coupled with some vibratory handpieces.

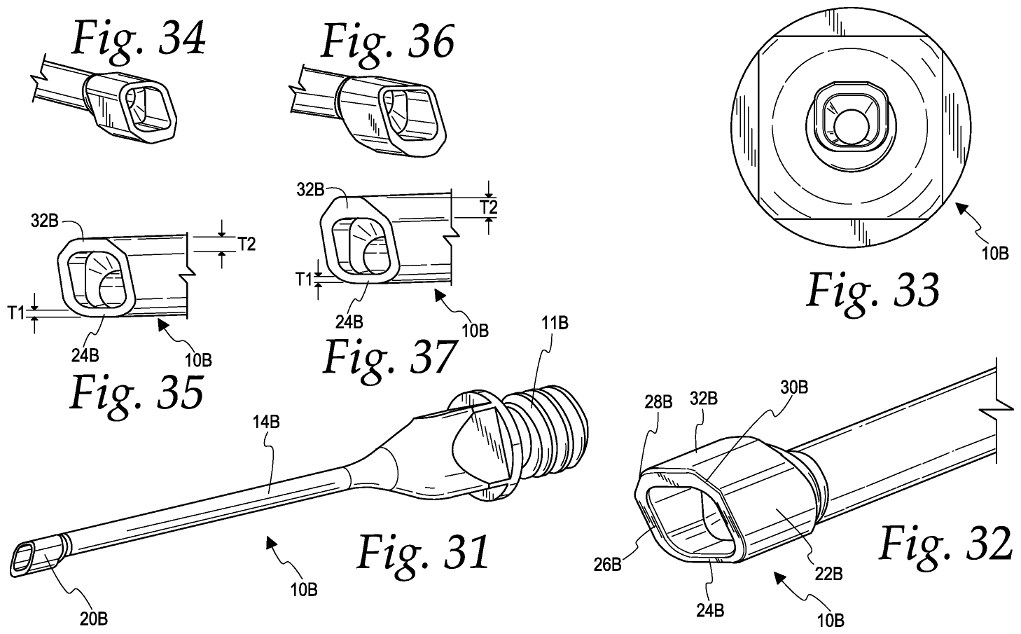

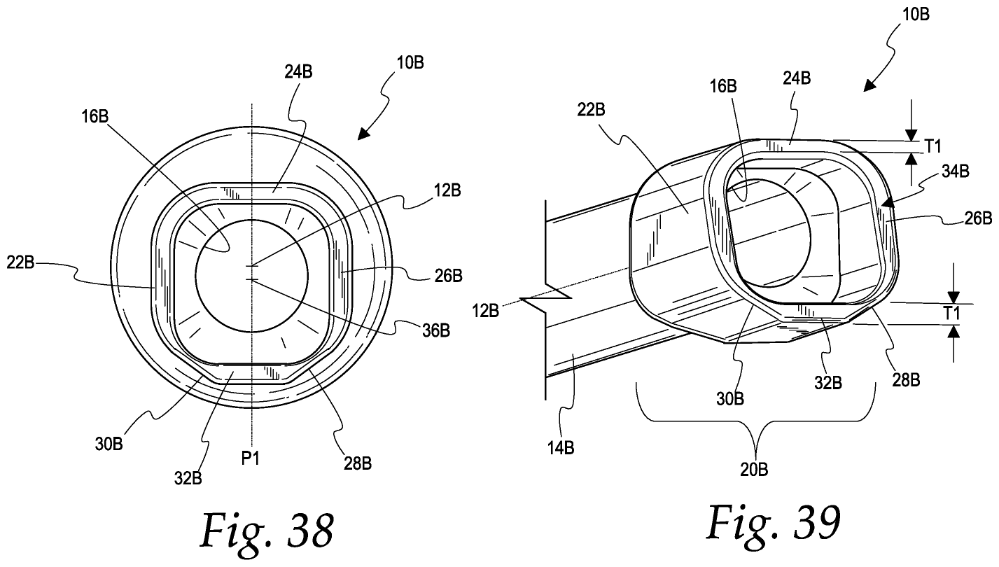

A third embodiment of a surgical needle according to the present invention is shown in FIGS. 31-39, designated by the numeral 10B, and functions similarly to the first illustrated embodiment of the needle 10 as previously described in FIGS. 1-15. The numbered features of the third embodiment of the needle illustrated in FIGS. 31-39 are analogous to features of the first embodiment of the needle that share the same number (without the suffix “B”). The third embodiment of the surgical needle 10B differs from the aforementioned first illustrated embodiment in that the needle 10B includes an emulsification tip 20B with six distinct walls 22B, 24B, 26B, 28B, 30B, 32B. With reference now to FIG. 38, a first pair of the walls 22B, 26B are substantially parallel to one another, a second pair of walls 24B, 32B are also substantially parallel to one another, and a third pair of walls 28B, 30B are angled with respect to one another, and extend or connect from one of the first pair to one of the second pair, as illustrated. The open mouth 34B of the tip portion 20B has a substantially square configuration, with rounded corners.

In some forms of the third embodiment of the needle 10B, illustrated in FIGS. 38 and 39, the walls 22B, 24B, 26B, 32B have a shared nominal thickness (T1) of about 100 microns, while the angled, converging walls 28B, 30B have a reduced thickness. In other forms of the third embodiment of the needle 10B, illustrated in FIGS. 31-37, the walls 22B, 24B, 26B have a shared nominal thickness (T1) of about 100 microns, while the offset or bottom wall 32B has a substantially greater thickness (T2) of between about 150 microns and about 250 microns for an enhanced wobble or chopping effect during operation.

The third illustrated embodiment of the needle 10B may provide a safer phacoemulsification as compared to the first illustrated embodiment of the needle 10 when coupled with some vibratory handpieces as a result of the truncated, converging walls 28B and 30B.

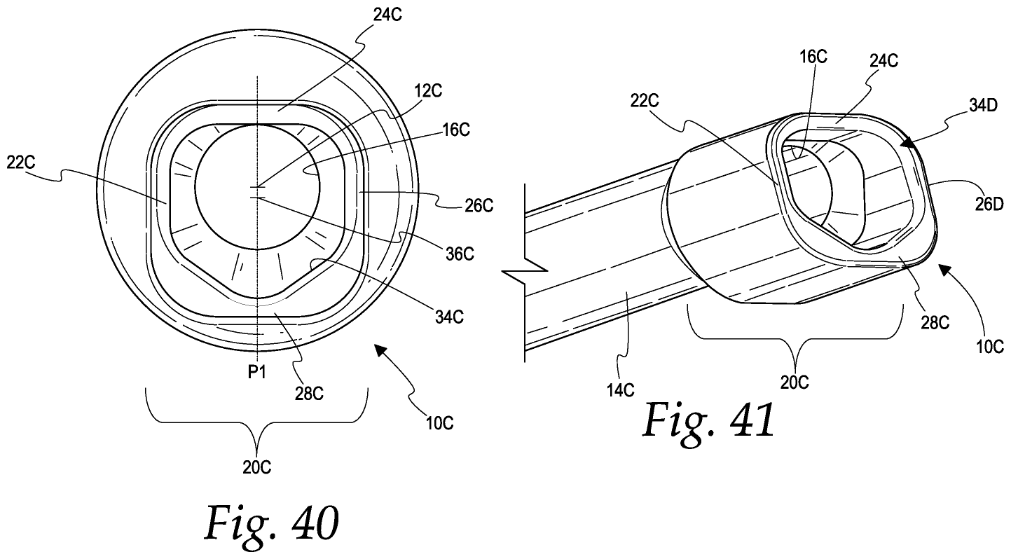

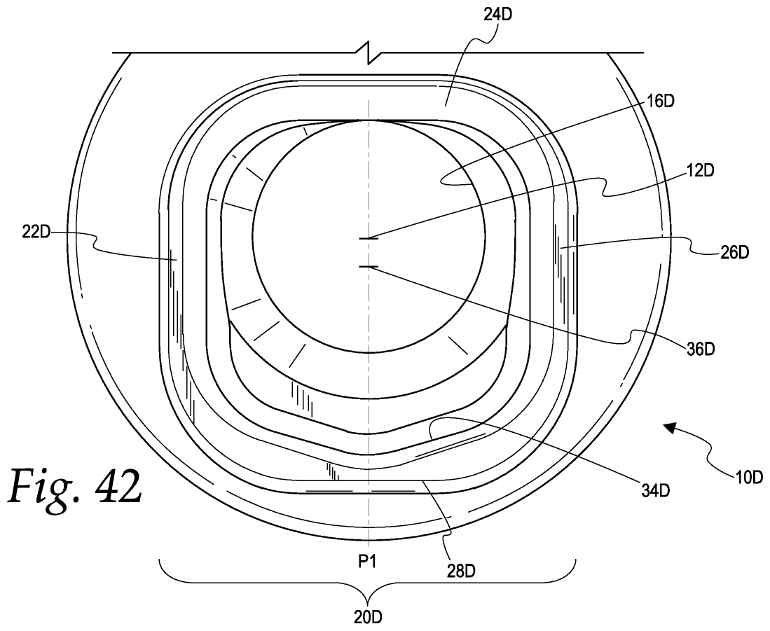

A fourth embodiment of a surgical needle according to the present invention is shown in FIGS. 40 and 41, designated by the numeral 10C, and functions similarly to the first illustrated embodiment of the needle 10 as previously described above. The numbered features of the fourth embodiment of the needle 10C illustrated in FIGS. 40 and 41 are analogous to features of the first embodiment of the needle that share the same number, but without the suffix “C”. The fourth embodiment of the surgical needle 10C differs from the aforementioned first illustrated embodiment in that the needle 10C includes an emulsification tip 20C that is substantially square, having four walls 22C, 24C, 26C, 28C which define a mouth 34C having an (internal) pentagonal configuration. It is believed that the needle 10C may be advantageous with respect to low cost, high quality manufacturability, as compared to the prior embodiments discussed above. The mouth 34C further defines a longitudinal mouth axis 36C that is parallel to, but offset from, the central longitudinal axis 12C of the needle shaft portion 14C.

With reference to FIG. 40, it can be seen that the open mouth 34C is symmetric about a plane P1 extending through both the longitudinal mouth axis 36C and the longitudinal shaft axis 12C. The corners of adjacent walls 22C, 26C in contact with the bottom most wall 28C of the tip portion 20C are substantially thicker than the remaining walls of the tip portion 20C to provide for the mouth 34C having a pentagonal configuration.

A fifth embodiment of a surgical needle according to the present invention is shown in FIG. 42, designated by the numeral 10D, and functions similarly to the fourth illustrated embodiment of the needle 10C as previously described immediately above. The numbered features of the fifth embodiment of the needle 10D illustrated in FIG. 42 are analogous to features of the fourth embodiment of the needle that share the same number, but without the suffix “D”. The fifth embodiment of the surgical needle 10D differs from the aforementioned fourth illustrated embodiment in that the needle 10D includes an emulsification tip 20D that defines corners of adjacent walls 22D, 26D in contact with the bottom most wall 28D which are substantially thinner than the preceding embodiment.

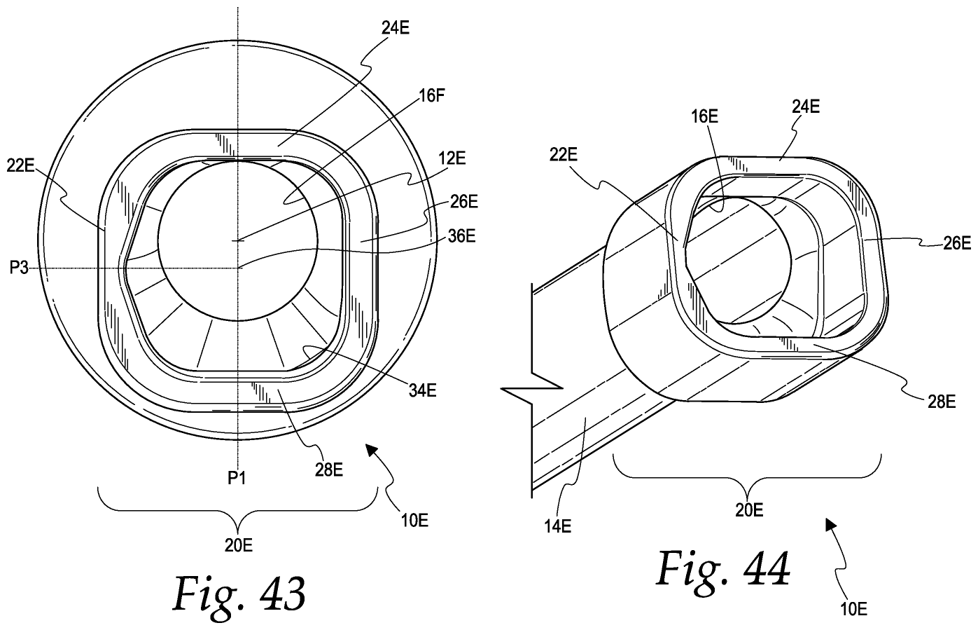

A sixth embodiment of a surgical needle according to the present invention is shown in FIGS. 43 and 44, designated by the numeral 10E, and functions similarly to the fifth illustrated embodiment of the needle 10D as previously described immediately above. The numbered features of the sixth embodiment of the needle 10E illustrated in FIGS. 43 and 44 are analogous to features of the fifth embodiment of the needle that share the same number, but without the suffix “E”. The sixth embodiment of the surgical needle 10E differs from the aforementioned fifth illustrated embodiment in that the needle 10E includes an emulsification tip 20E with four side walls 22E, 24E, 26E, 28E defining an open mouth 34E that is pentagonal in shape but lacking symmetry about the plane P1 that extends through both the longitudinal mouth axis 36E and the longitudinal shaft axis 12E. Instead, the mouth 34E has symmetry about a transverse horizontal plane P3, which is normal to the plane P1. This arrangement facilitates efficient use, and may be particularly suitable for use with certain vibratory instruments or handpieces.

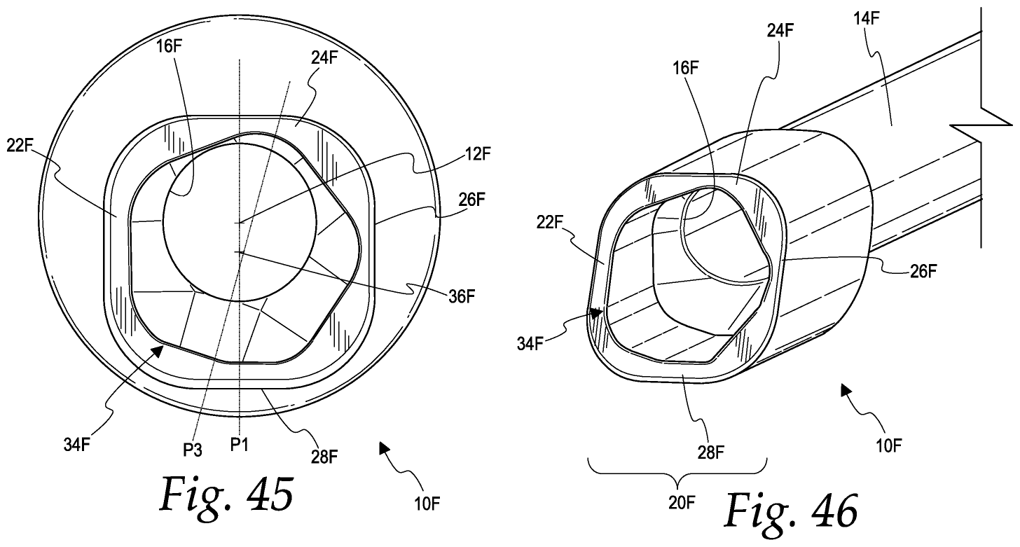

A seventh embodiment of a surgical needle according to the present invention is shown in FIGS. 45 and 46, designated by the numeral 10F, and functions similarly to the sixth illustrated embodiment of the needle 10E as previously described immediately above. The numbered features of the seventh embodiment of the needle 10F illustrated in FIGS. 45 and 46 are analogous to features of the sixth embodiment of the needle that share the same number, but without the suffix “F”. The seventh embodiment of the surgical needle 10F differs from the aforementioned sixth illustrated embodiment in that the needle 10F includes an emulsification tip 20F defining an open mouth 34F that is pentagonal in shape and having symmetry about a transverse plane P3, which is not normal to the plane P1.

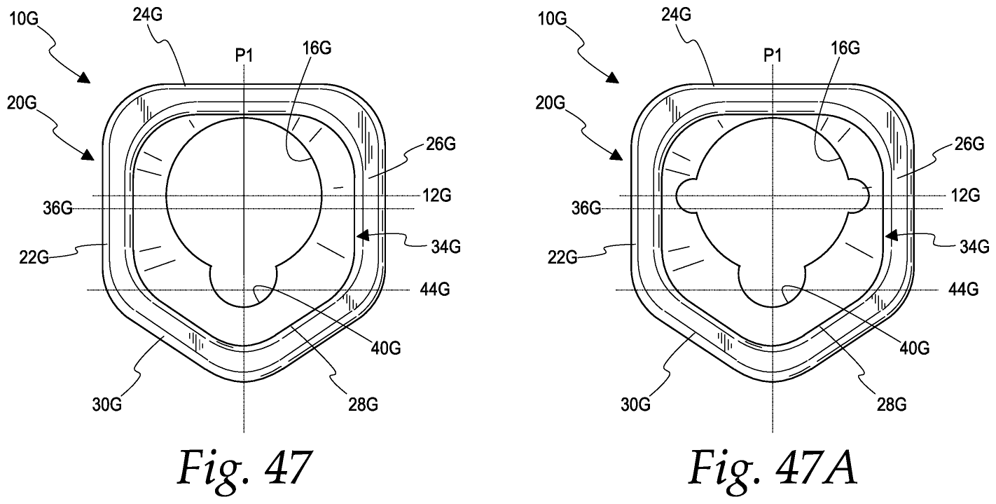

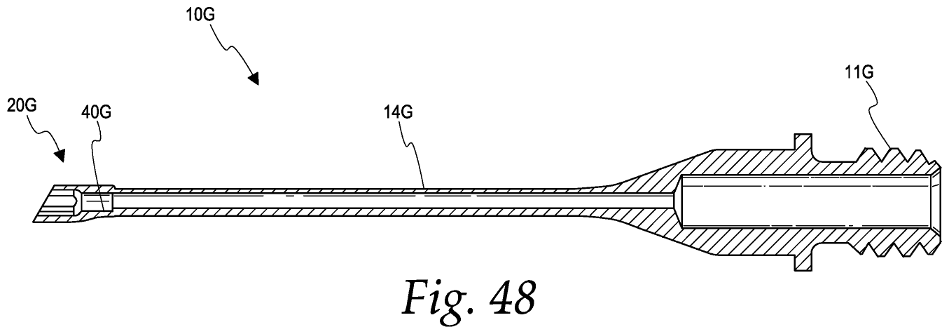

An eighth embodiment of a surgical needle according to the present invention is shown in FIGS. 47 and 48, designated by the numeral 10G, and functions similarly to the first illustrated embodiment of the needle 10 as previously described above. The numbered features of the eighth embodiment of the needle 10G illustrated in FIGS. 47 and 48 are analogous to features of the first embodiment of the needle that share the same number, but without the suffix “G”. The eighth embodiment of the surgical needle 10G differs from the aforementioned first illustrated embodiment in that the needle 10G includes an emulsification tip 20G defining an intermediate passage 40G that is offset from the aspiration passage 16G of the needle body 14G (FIG. 48 only). The intermediate passage 40G creates a weighted effect in the tip portion 20G to enhance and/or tune the wobble or imbalance during vibration of the needle 10G. The intermediate passage 40G in the tip portion 20G is preferably drilled in a secondary step from the drilling of the aspiration passage 16G of the needle body 14G. The intermediate passage 40G preferably has the form of a circular or semi-circular trough centered along an axis 44G that is offset from the central axis 12G of the aspiration passage 16G along a central vertical plane P1. However, it will be understood that the intermediate passage 40G may have other cross-sectional shapes, such as square, elliptical, triangular, other polygonal or irregular shape. A variation of the eighth embodiment of the needle 10G is illustrated in FIG. 47A, wherein the tip portion 20G includes two additional intermediate passages 40G located on either side of the plane P1, each of which has a semi-circular shape.

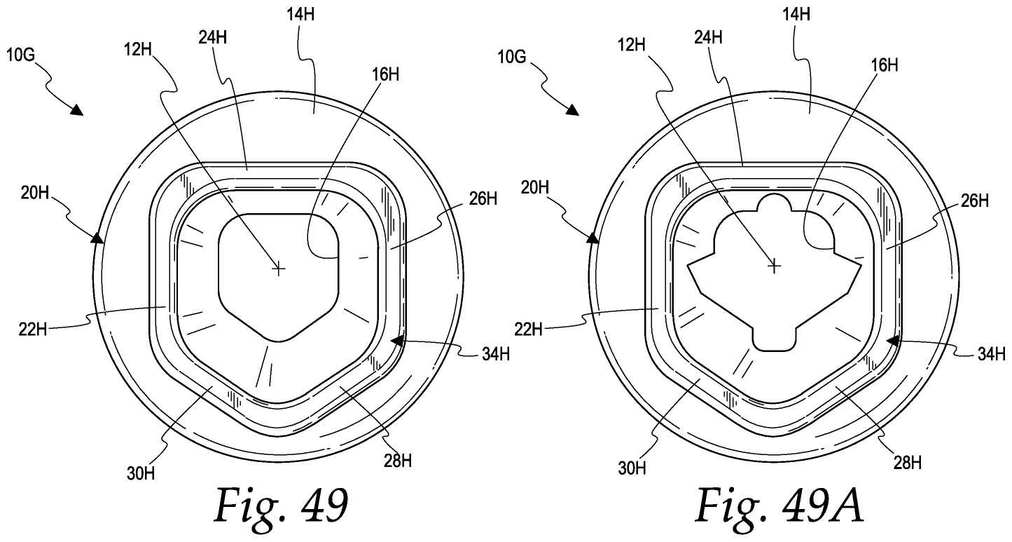

A ninth embodiment of a surgical needle according to the present invention is shown in FIG. 49, designated by the numeral 10H, which functions similarly to the first illustrated embodiment of the needle 10 as previously described above. The numbered features of the ninth embodiment of the needle 10H illustrated in FIG. 49 are analogous to features of the first embodiment of the needle that share the same number, but without the suffix “H”. The ninth embodiment of the surgical needle 10H differs from the aforementioned first illustrated embodiment in that the needle 10H includes an aspiration passage 16H of the needle body having a pentagonal cross-sectional shape inset from the pentagonal cross-sectional shape of the walls 22H, 24H, 26H, 28H, 30H of the tip 20H. The aspiration passage 16H defines a central axis that is offset from the central axis of the mouth defined by the walls 22H, 24H, 26H, 28H, 30H. A variation of the ninth embodiment of the needle 10H is illustrated in FIG. 49A, wherein the tip portion 20H includes two circular (top and bottom) and two pentagonal (left and right) intermediate passages located on either side of the vertical plane.

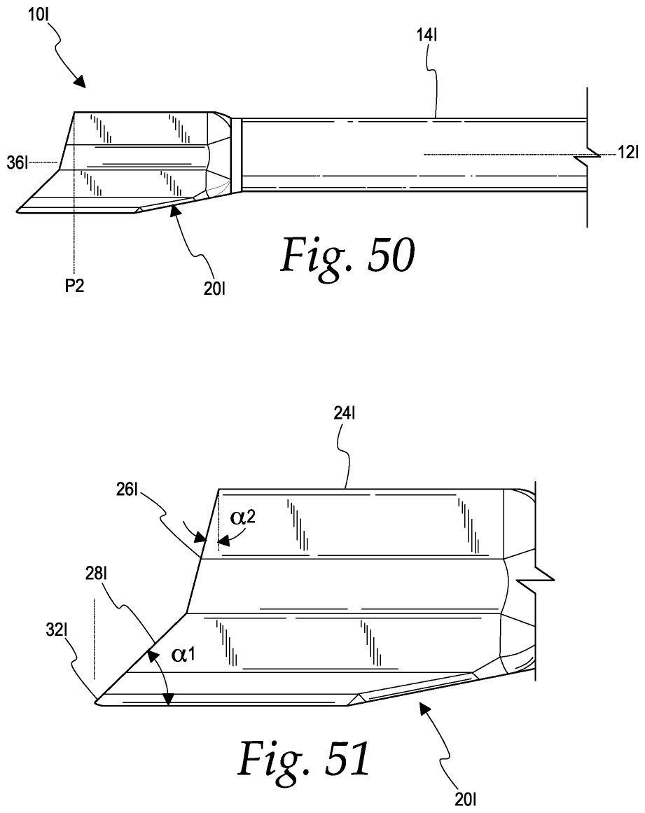

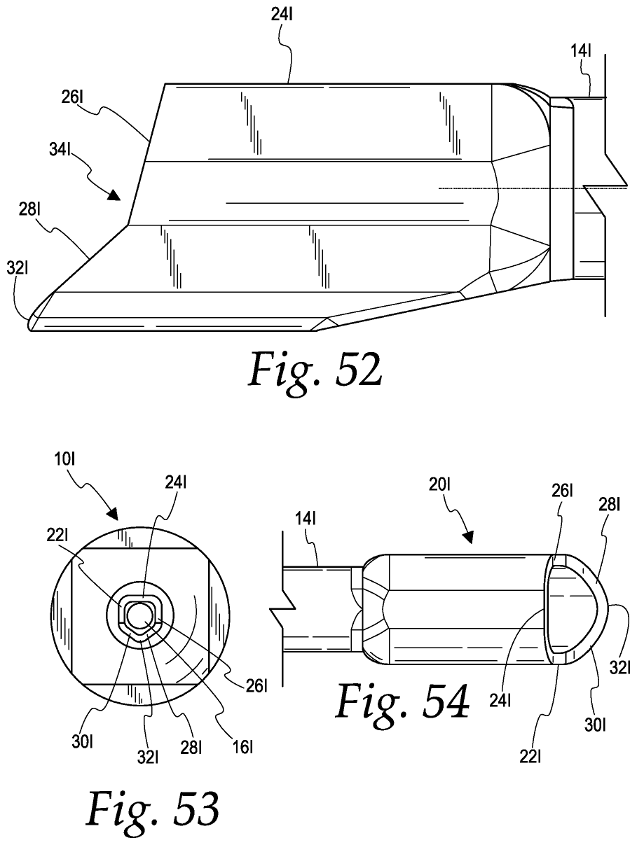

A tenth embodiment of a surgical needle according to the present invention is shown in FIGS. 50-54, designated by the numeral 10I, which functions similarly to the first illustrated embodiment of the needle 10 as previously described above. The numbered features of the tenth embodiment of the needle 10I illustrated in FIGS. 50-54 are analogous to features of the first embodiment of the needle that share the same number, but without the suffix “H”. The tenth embodiment of the surgical needle 10I differs from the aforementioned first illustrated embodiment in that the needle 10I includes a tip portion 20I having a pentagonal cross-sectional shape inset from the pentagonal cross-sectional shape of the walls 22I, 24I, 26I, 28I, 30I, wherein the lower or bottom walls 28I and 30I extend outwardly of at least the upper wall 24I at a different angle α1 relative to a vertical plane P2 extending normal to the central axis 12I of the needle body 14I and the central axis 36I of the offset tip portion 20I as compared to the angle α2 of the upper walls 22I and 26I relative to the plane P2. Preferably, the lower or bottom walls 28I and 30I are angled about 45 degrees relative to the plane P2, while the upper side walls 22I and 26I are angled about 15 degrees relative to the plane P2. The vertex of walls 28I and 30I meet in a sharpened point 32I. The inventors believe that the steep angle of the lower walls 28I and 30I, relative to the upper walls of the tip portion may provide enhanced trenching of the nucleus. In some forms, the tip portion 20I may have seven walls in a heptagonal configuration for improved trenching. A variation of the tenth embodiment of the needle 10I is illustrated in FIGS. 52-54, wherein the tip portion 20I includes a blunted, rounded vertex portion 32I between the lower or bottom walls 28I and 30I.

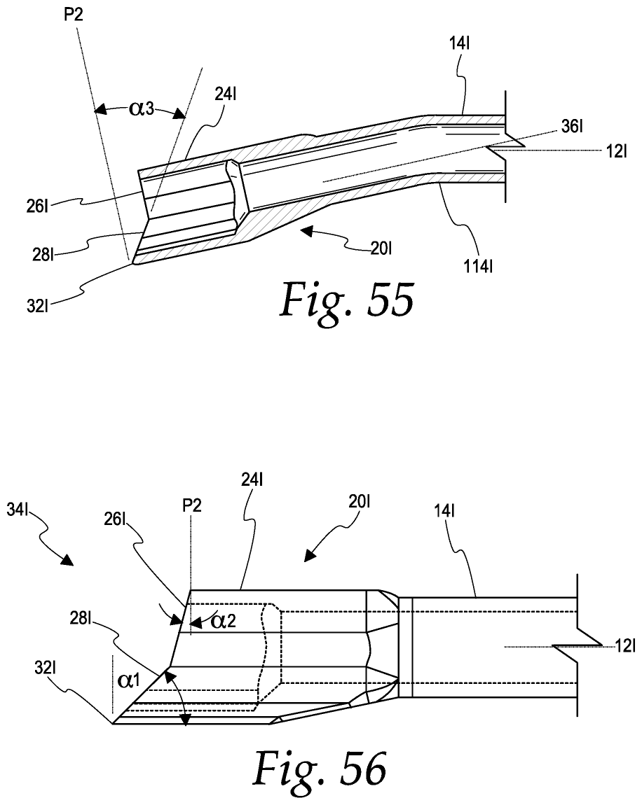

Another variation of the tenth embodiment of the needle 10I is illustrated in FIG. 55, wherein the lower or bottom walls 28I and 30I extend axially outwardly of the upper side walls 22I and 26I at an angle α3 of about 30 degrees (+/−3 degrees) relative to a vertical plane P2 extending normal to the central axis 36I of the offset tip portion 20I. Furthermore, the angle α2 (not designated) of the upper walls 22I and 26I relative to the plane P2 is about 0 degrees in this embodiment of the needle 10I. It can be seen that the needle 10I includes a bend 114I located proximate the tip portion 20I and the needle body 14. In some forms, not illustrated, the angle α3 may be between 0 degrees (planar or flat distal end of the tip) to 90 degrees (perpendicular to the plane P2).

Still another variation of the tenth embodiment of the needle 10I is illustrated in FIG. 56, wherein the lower or bottom walls 28I and 30I extend outwardly of at least the upper wall 24I at a different angle α1 relative to a vertical plane P2 extending normal to the central axis 12I of the needle body 14I and the central axis 36I of the offset tip portion 20I as compared to the angle α2 of the upper walls 22I and 26I relative to the plane P2. Preferably, the lower or bottom walls 28I and 30I are angled about 50 degrees (+/−3 degrees) relative to the plane P2 (or about 40 degrees (+/−3 degrees) relative to the plane normal to P2 as illustrated), while the upper side walls 22I and 26I are angled about 15 degrees (+/−3 degrees) relative to the plane P2. In some forms, not illustrated, the angle α2 may be between 0 degrees (planar or flat distal end of the tip) to 90 degrees (perpendicular to the plane P2), while the angle α1 may be between 0 degrees (planar or flat distal end of the tip) to 90 degrees (perpendicular to the plane P2) and any combination of said ranges of α1 and α2.

The inventors have found that the needle designs described herein have exhibited better followability of the nucleus, better fluidics, no observable chatter, repulsion, or bubbles regardless of nucleus grade, no milk-like fluid when emulsifying a grade 4 or 5 nucleus, improved sculpting or grooving of the nucleus, improved chamber stability, and better overall efficiency when compared to prior art phacoemulsification needles. It is currently believed that the pentagonal or pentagonal-like shape of the needle tip portions describe above creates a vortices to increase the volume of inflow of balanced salt solution and emulsified nucleus in the tip has increased followability.

From the foregoing, it will be observed that numerous modifications and variations can be effected without departing from the true spirit and scope of the novel concept of the present invention. It is to be understood that no limitation of the broadest concepts with respect to the specific embodiments illustrated herein is intended or should be inferred. The disclosure is intended to cover, by the appended claims, all such modifications as fall within the scope of the claims.

1. A phacoemulsification needle, comprising:
a needle shaft portion defining a longitudinal shaft axis, and having an internal aspiration passage; and
an emulsification tip joined to a distal end of said needle shaft portion,
said emulsification tip having five side walls defining a perimeter of an open mouth communicating with said aspiration passage, three of said side walls defining a first thickness, the remaining two of said side walls converging at a location in a vertical plane that extends through said longitudinal shaft axis and having a second thickness that is greater than said first thickness.

2.-11. (canceled)

12. A phacoemulsification needle, comprising:
a needle shaft portion defining a longitudinal shaft axis, and having an internal aspiration passage; and
an emulsification tip joined to a distal end of said needle shaft portion,
said emulsification tip having five side walls defining a perimeter of an open mouth communicating with said aspiration passage, two of said side walls converging in a rounded corner at a location in a vertical plane that extends through said longitudinal shaft axis.

13. The phacoemulsification needle in accordance with claim 12 wherein said five side walls have the same thickness.

14. The phacoemulsification needle in accordance with claim 13, wherein said thickness is about 100 microns.

15. The phacoemulsification needle in accordance with claim 12 in combination with a vibratory handpiece.

16. The phacoemulsification needle in accordance with claim 12,
wherein said open mouth defines a longitudinal mouth axis that is parallel to, and offset from, said longitudinal shaft axis.

17.-21. (canceled)

22. A phacoemulsification needle, comprising:
a needle shaft portion defining a longitudinal shaft axis, and having an internal aspiration passage; and
an emulsification tip joined to a distal end of said needle shaft portion,
said emulsification tip having four side walls defining a perimeter of an open mouth communicating with said aspiration passage, said open mouth having a pentagonal shape.

23.-61. (canceled)

62. The phacoemulsification needle in accordance with claim 12 wherein three of said side walls define a first thickness, the remaining two of said side walls converge at a location in a vertical plane that extends through said longitudinal shaft axis and have a second thickness that is greater than said first thickness.

63. The phacoemulsification needle in accordance with claim 62 wherein said first thickness is about 100 microns and wherein said second thickness is between about 150 and about 170 microns.

64. The phacoemulsification needle in accordance with claim 12 wherein said emulsification tip is sandblasted.

65. The phacoemulsification needle in accordance with claim 12 wherein adjacent ones of said five side walls converge at convex corners.

66. The phacoemulsification needle in accordance with claim 12 wherein said aspiration passage has a pentagonal cross-sectional shape in a plane normal to said longitudinal shaft axis.

67. The phacoemulsification needle in accordance with claim 12 wherein said emulsification tip has a distal edge defined by a converging pair of said walls disposed at a first acute angle to a plane extending normally through said longitudinal shaft axis, and a second pair of said walls are disposed at a second acute angle relative to said plane, said first acute angle being greater than said second acute angle.

68. The phacoemulsification needle in accordance with claim 12 wherein said emulsification tip has an intermediate passage formed therein that is offset from said longitudinal shaft axis.

69. The phacoemulsification needle in accordance with claim 68 wherein said intermediate passage is semi-circular and is centered along a vertical plane extending through said longitudinal shaft axis.

70. The phacoemulsification needle in accordance with claim 12 wherein said emulsification tip has a distal edge disposed at an acute angle to a plane extending normally through said longitudinal axis of said needle shaft portion.

71. The phacoemulsification needle in accordance with claim 70 wherein said acute angle is between about 20 and 40 degrees.

72. A phacoemulsification needle, comprising:
a needle shaft portion defining a longitudinal shaft axis, and having an internal aspiration passage; and
an emulsification tip joined to a distal end of said needle shaft portion,
said emulsification tip having at least five side walls defining a perimeter of an open mouth communicating with said aspiration passage, wherein said emulsification tip has a distal edge defined by a converging pair of said walls disposed at a first acute angle to a plane extending normally through said longitudinal shaft axis, and a second pair of said walls are disposed at a second acute angle relative to said plane, said first acute angle being greater than said second acute angle.
